(19) United States
(12) Patent Application Publication
(10) Pub. No.: US 2013/0172735 A1
(43) Pub. Date: Jul. 4, 2013

(54) OPTICAL COHERENT IMAGING MEDICAL DEVICE

(71) Applicants: Marc Andre, Spiegel b. Bern (CH); Michael Friedrich, Vevey (CH); Andrey Naumenko, Ecublens (CH); Romain Farkas, Lausanne (CH); Theo Lasser, Denges (CH)

(72) Inventors: Marc Andre, Spiegel b. Bern (CH); Michael Friedrich, Vevey (CH); Andrey Naumenko, Ecublens (CH); Romain Farkas, Lausanne (CH); Theo Lasser, Denges (CH)

(73) Assignee: AIMAGO S.A., Lausanne (CH)

(21) Appl. No.: 13/636,268

(22) PCT Filed: Mar. 16, 2011
(86) PCT No.: PCT/IB2011/051098

§ 371 Date: Mar. 4, 2013

(30) Foreign Application Priority Data
Mar. 26, 2010 (IB) PCT/IB2010/051332

(51) Int. Cl. A61B 6/00; A61B 5/00

(52) U.S. Cl. CPC A61B 6/4405; A61B 5/0073
USPC 600/425

(57) ABSTRACT

The invention concerns an OCI medical device (100) comprising the following elements:—a coherent light source (120),—a 2D light sensor (120),—a screen (110) that displays OCI map and/or mixture map,—a processing unit that calculates the OCI map; all said elements being included in a single movable unit. The invention also relates to the use of said OCI medical device.

### FIELD OF INVENTION

The invention relates to the use of Optical Coherent Imaging (OCI) in the medical field and more precisely to the use of medical devices using this optical tool.

### DEFINITIONS

The following terms and related definitions are used in the present text.

“camera unit” is the physical unit where, as presented in this invention, the visualization screen and the OCI optics, including OCI sensor, illumination and other optics are implemented. This unit usually is moved as a whole.

“hand-movable camera” is a camera unit that can be moved by the user with little force. Usually the hand-movable camera is mounted to a supporting arm.

“OCI map” is any digital two or three dimensional data that is extracted from the optical coherent imaging. An example is the perfusion extracted from LDI. For 2-dimensional data it can be used directly as an image with x-y coordinates. 3-dimensional data can be reduced to 2D using projection or slicing. The OCI map as used in this document can also be a fOCI map.

“fOCI map” is a functional imaging result as disclosed for instance in WO 2010/004364. This definition matches the definition given in that patent application.

“white-light image” is any still or video image created with the use of an image sensor (CMOS, CCD or other) and being displayed as such, thus without further intensive processing other than filtering and improving the image quality. The image can either be color or black/white. As an example: all digital camera and video equipment produce a white-light image. The term white-light was introduced to show the difference to an optical coherent image or OCI map. It shall not be limited to pure white-light illumination but also include image from other color light source that was taken as standard photo.

“real-time” applied to visualization is a reasonable visualization update frequency with short latency so that the user can have an immediate feedback from any change of the observed area or the movement of the camera.

“confidence level” is any calculated or measured characteristic of an OCI map that summarizes the confidence with regard to the correct OCI map value for a given pixel, for a given region or for the overall OCI map. Example factors that can be reflected in the confidence level are imaging sharpness, curvature effects, camera stability, and noise.

“display” and “screen” shall be used as synonyms.

“aperture” shall be referenced as the entry/exit point(s) of the OCI optical path to the camera unit.

### STATE OF THE ART

There exists a number of imaging techniques in the medical field. X-ray based imaging, MRI, ultrasound imaging are proven techniques that are commonly used. Another group of imaging techniques is Optical Coherent Imaging (OCI).

Optical coherent imaging is a non-contact imaging utilizing, to a large extent, the physical properties and in particular the coherence properties of light. This imaging modality integrates a detector technology, combined with an appropriate coherent light source and an image processing unit for extracting the flow characteristics of the observed body area of interest. Thereby, it allows the diagnosis or observation of multiple diseases and disorders such as peripheral vascular diseases, skin irritations, diabetes, burns, organ transplants, tissue grafts and even functional brain imaging. This method is non-invasive because it involves no physical contact; therefore risk of infection and discomfort are greatly avoided.

Sub-classes of OCI include, but are not limited to: Laser Doppler Imaging (LDI), Laser Doppler Spectroscopic Imaging (LDSI), Laser Speckle Imaging (LSI), Optical Coherence Tomography (OCT), Functional Optical Coherent Imaging (fOCI).

Existing OCI medical devices, like many other medical imaging tools, are at least partially static. The OCI sensors may be movable but the screen is permanently fixed to a support.

It would be therefore convenient for the user, in one single movement, to simultaneously orient the OCI medical device on an observed area and view this area.

### GENERAL DESCRIPTION OF THE INVENTION

The present invention provides a solution to the problem mentioned in the previous chapter.

It concerns an OCI medical device comprising the following elements:
a coherent light source,
a 2D light sensor,
a screen that displays OCI map and/or mixture map;
   all said elements being included in a single movable unit.

Preferred embodiments of the invention are listed in the claims.

Advantageously the device is a hand-movable camera unit featuring a display that works as a virtual window. The display allows for the real-time visualization of OCI. The display also allows for the OCI-enhanced real sight visualization. Handling of the device is very natural to the user because he just moves the virtual window to the region of interest and sees on the screen the OCI map. If the device was fully transparent he would see the same region with direct eye view.

One of advantages of the invention is to provide a so far unachievable improvement in usability of an OCI medical device. It comprises OCI acquisition tools and visualization display within a single, preferably hand-movable housing. The invention also provides other innovative improvements that will be discussed later in this document.

The invention also reduces the learning time for the user significantly. The user can move the virtual window over the patient. In this window the user doesn't just see a picture of the surface but also a real-time visualization of body parameters. The body parameter can be blood perfusion, blood concentration, blood speed, any fOCI map or other body functions of interest taken with the technique of Optical Coherent Imaging.

The invention not only provides a handheld but also a hand-movable device. This considerably facilitates the user's work. By “hand-movable” it is meant a mobile device that can be moved by hands during its use but that, unlike a handheld device, can have some kind of support (e.g. a stand with a mechanical arm, a ceiling or wall mounted suspension, or some other). Such a support holds its corresponding hand-movable device while the device is used. Thus with the present invention users do not need to hold the device while using it, having their hands free for other manipulations.

The invention offers in particular the following advantages with regard to the existing OCI systems:
Easy handling and focusing of the device;
Shorter learning time to use the device;
Overlooking the area of interest with a direct eye view and with an OCI-enhanced view simultaneously, with a user having free hands available for other manipulations at the same time;
No dead angles while viewing the area of interest

### DETAILED DESCRIPTION OF THE INVENTION

The invention will be better understood through non-limitative examples that are illustrated by the following figures:

FIG. 1 shows a side view of a proposed Embedded OCI system.

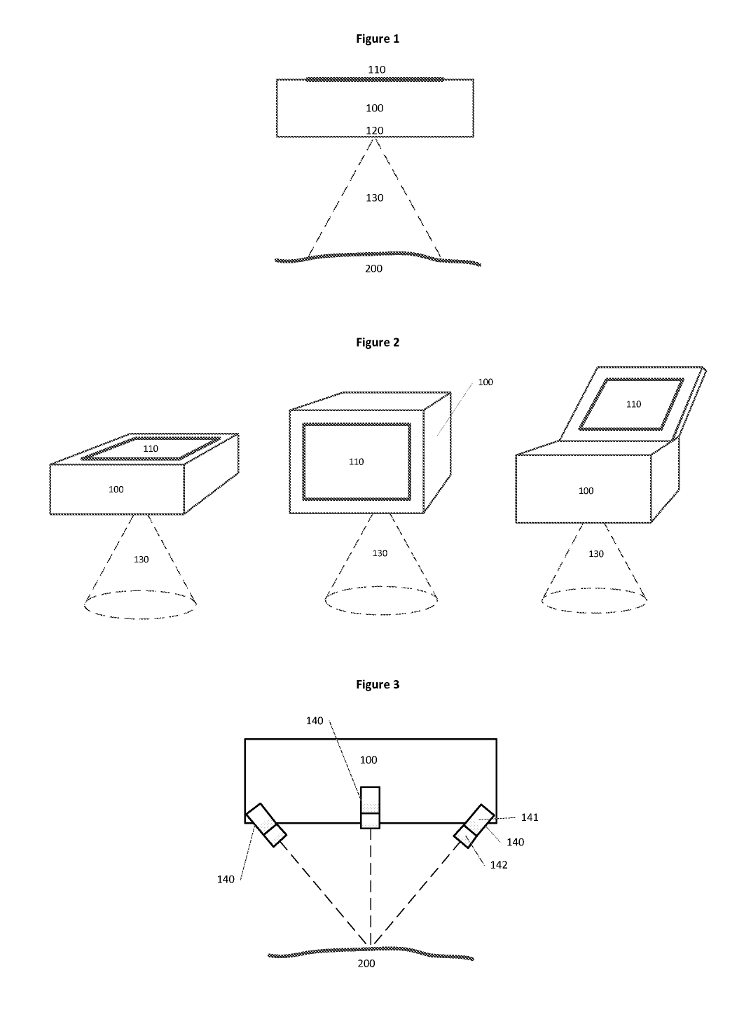

FIG. 2 shows pseudo perspective views of possible mounting of the screen to the camera

FIG. 3 shows the simple focus laser indication system with 3 collimated light sources

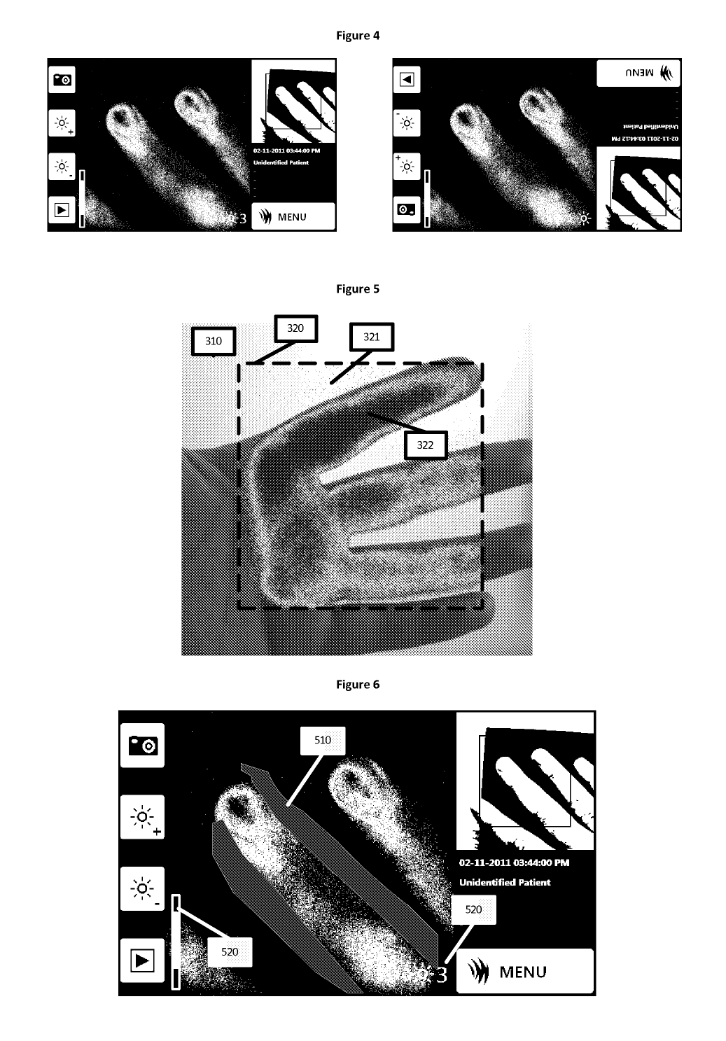

FIG. 4 screenshots of an Embedded OCI system with user interface rotation for working on two sides of the device

FIG. 5 shows mixture map of an LDI perfusion flow over the white-light image.

FIG. 6 shows an example of overlay over the OCI map with additional indications for the user.

As shown on FIG. 1 the screen (110) is part of the same unit (camera unit, 100) as the OCI optics (120). If the user moves the camera unit he automatically also moves the screen for a similar distance. The OCI optics consists of laser(s), 2D light sensor(s) and possibly other optical and mechanical elements. A 2D light sensor is an image sensor of any technology that can work with visible, infrared or ultraviolet light. The OCI optics aperture (or 2D light sensor aperture) is the entry point of the optics path into the camera unit. In the best case the screen is on the opposite side of the aperture. The OCI optics with its optics path (130) visualizes a body parameter of the observed area of the patient (200). In addition a standard white-light camera can observe the same observed area, either through the same or a similar optical path. The size of the observed area for OCI and white-light can be different.

For the completeness it shall be mentioned that the screen can also be attached on other positions of the camera. Possible positions are the side or any other angle. FIG. 2 shows some examples. For the usability reasons mentioned in the background discussion, in most cases the camera unit is also mounted to a supporting arm. In that case the screen may also be mounted to that arm as long as it is in close proximity of the OCI optics and moves together with the OCI optics.

A processing unit further processes the data obtained from the OCI sensor and calculates the OCI maps. There are different processing techniques available to people skilled in the art and the processing shall not be part of this invention. Processing is done using CPU, DSP, FPGA, ASIC or a combination of those. The processing unit can either be a part of the camera unit or a separate unit with a wired or a wireless connection.

On the display an OCI map, a white light image or a combination of both is shown in real-time. The orientation of the image matches the orientation of the visualized object. This means that the user sees an object in the same orientation on the screen as he would see it without the screen with a direct eye view. In optimal case the observed surface is shown in a 1:1 scaling thus the image on the screen has roughly the size of the observed area, but the scaling can be different without limiting the invention.

OCI is a non-contact technology and has to work at some reasonable working distance of a few centimeters up to a meter, while the best working distance for usability reasons ranges from 15 cm to 30 cm. In order to find the focus a simple focus indication system can be implemented. A pattern is projected to the observed skin area which has a distance depending scale and/or shape. This pattern indicates if the camera is within working distance. In addition it is also possible to show if the camera is too close or too far with regard to the focal distance.

A possible implementation of this focus indication system is shown in FIG. 3. At least 2 collimated light sources (140) consisting of a light source (141) and a collimating optics (142) are placed on different positions of the camera unit (100). These collimated light sources project a small point on the observed surface (200). The small points collide in the focus distance of the camera unit. In the best case the collision point is also in the center of the observed area. With this simple system the user has to bring the camera in such a distance that the two points are projected on the same spot on the observed area and thus only a single projected point is visible. When adding a 3^{rd} laser it is also possible to show if the camera is too close or too far from the observed surface with regard to the focal distance. Because the projected triangular pattern changes its orientation when camera unit passes through its focal point. With good real-time characteristics of the OCI system this is not necessary, because the user determines with the dynamics of imaging if the camera unit is too far or too close.

A good color for the focus indication light is green because this is well seen on the skin. In addition most OCI systems use infrared lasers and infrared detectors. It is easy to filter the green focus indication light from being captured by the OCI detector using band-pass, long-pass filter, dichroic mirror or any other filter known to those skilled in the art.

The screen acts as HMI (human machine interface). Its primary function is to show an OCI map, a white light image or a mixture map. It is also possible to show multiple maps at the same time. In the best case the user can switch between different maps or map combinations. The secondary functions of the screen are: to allow users to configure the system and to show other information needed for functioning of the device.

Especially in operating rooms an OCI device may be used by two doctors working on opposite sides of the patient. Another aspect of this invention is the rotation or swapping of the user interface. Rotating or swapping involves all user interface parts on the screen but it doesn't necessary mean that the user interface layout remains exactly the same. FIG. 4 gives an example. In addition the OCI map and white-light image do not turn, because their orientation with regard to the user shall remain, as described in the paragraphs above. The layout may be differently designed in different orientations because of some external constraints. In the example given in FIG. 4 this is the location of the buttons on the left, because the device has adjacent physical buttons.

The mixture map, as presented in this invention, is an overlay of an OCI map over the white-light image. In order to achieve a good image, irrelevant values of the OCI map can be removed. For perfusion these would be the non- or very low-perfused values. These values are made fully transparent while the others are shown with some transparency (α). The transparency can either be a uniform fixed; a user-set uniform value or it can be calculated depending on the value (v) and/or the confidence level (γ) of the OCI map, again with some user-configurable level. Of course other approaches or extensions, like taking neighboring pixels into the formula, are possible and shall not limit this invention.

α=cᵤₛₑᵣ Uniform transparency

α_{xy}=cᵤₛₑᵣ*f(v_{xy},γ_{xy}); Non-uniform transparency

with
v_{xy} being the OCI map value at coordinate (x,y)

α_{xy,y}=cᵤₛₑᵣ*f(v_{1,all},v_{2,all}, . . . , γ₁ₐₗₗ,γ₂ₐₗₗ, . . . x,y); Non-uniform transparency, extended

with
v_{1,all} being all data of a OCI map and v_{2,all} being all data of a different OCI map

FIG. 5 shows an example implementation where the perfusion of a finger is shown. In this example the white-light image (310) with a larger observed area is overlayed with an OCI map (320, border not visible to user). The non- and low-perfused values are set to fully transparent (321) while the other values don't have any transparency (322).

The image can further be extended with additional overlay information consisting of text, figures or drawings. This can be, but not limited to, the highlighting of interesting spots, the labeling of values or settings (530), indicating color bar (520) or the cover of regions with low confidence level (510). FIG. 6 shows an example.

The user interaction with the OCI system, according to this invention, is by touch screen, by physical buttons, by foot pedal or remote buttons or by any combination of those. The touch screen technology can be resistive, capacitive or any other technologies known to those skilled in the art.

In the preferred way the device is small such that the user can view the screen and the observed area with direct eye view simultaneously. For that reason the screen should have a good viewing angle.

It is also possible to draw features like outline of the observed skin area, regions with some OCI map values above or below a threshold, or other data mentioned above for overlay information directly to the skin of the patient. This drawing is done using light projection or laser illumination. It can be single or multi-color. In the best case the user can select to enable or to disable the projection. There are many projection technologies known to people skilled in the art.

1. An OCI medical device comprising the following elements:
a coherent light source,
a 2D light sensor,
a screen that displays OCI map and/or mixture map;
all said elements being included in a single movable unit.

2. An OCI medical device according to claim 1 wherein said unit comprises an upper face and a bottom face, said screen being located on said upper face and said 2D light sensor and/or its aperture being located on said bottom face.

3. An OCI medical device according to claim 1 wherein said unit comprises a front face and a bottom face, said screen being located on said front face and said 2D light sensor and/or its aperture being located on said bottom face.

4. An OCI medical device according to claim 1 wherein said unit comprises a rotatable panel and a bottom face, said screen being located on said rotatable panel and said 2D light sensor and/or its aperture being located on said bottom face.

5. An OCI medical device according to claim 1 comprising two collimated coherent light sources which are oriented in a manner as to focus their respective beams on a same point.

6. An OCI medical device according to claim 1 comprising real-time OCI means which are adapted to show on said screen a real-time OCI of an observed area.

7. An OCI medical device according to claim 1 comprising real-time color or black-and-white visualization means which are adapted to show on said screen a real time regular view of an observed area.

8. An OCI medical device according to claim 6 showing a mixture map wherein the transparency of the OCI map is either a uniform fixed, a user-set uniform value or calculated depending on the value and/or the confidence level of the OCI map, optionally with some user-configurable level.

9. An OCI medical device according to claim 6 that allows the user interface to be turned or swapped while keeping the said real-time OCI means or the said real-time color or black-and-white visualizations means oriented with the real object.

10. An OCI medical device according to claim 1 that allows a user to simultaneously see an observed area through said screen and with a direct eye view.

11. An OCI medical device according to claim 1 wherein said device is portable.

12. An OCI medical device according to claim 11 which is hand-movable and has a support from which it can be detached.

13. Use of an OCI medical device as defined in claim 1, said device comprising sources of visible light beams that form a projection on an observed area with a distance-dependent scale of the projected pattern, in a way as to allow to determine the focal distance of the OCI optics at the position where the projected pattern scales down to a single point.

14. Use of an OCI medical device as defined in claim 9 where the user can operate the device from at least two sides while having the user interface oriented with the user and the said real-time OCI means or the said real-time color or black-and-white visualizations means oriented with the real object.
